SEQUENCE LISTING (28 sequences)

SEQ ID NO: 1 - protein, 8 aa, synthetic construct
GYSITSDY

SEQ ID NO: 2 - protein, 5 aa, synthetic construct
SYIGS

SEQ ID NO: 3 - protein, 8 aa, synthetic construct
TIRNYFDY

SEQ ID NO: 4 - protein, 11 aa, synthetic construct
SASQGISNYLN

SEQ ID NO: 5 - protein, 7 aa, synthetic construct
DTSSLHS

SEQ ID NO: 6 - protein, 9 aa, synthetic construct
QQYSNFPXT

SEQ ID NO: 7 - protein, 7 aa, synthetic construct
GFNIKDY

SEQ ID NO: 8 - protein, 6 aa, synthetic construct
DPENGN

SEQ ID NO: 9 - protein, 9 aa, synthetic construct
YGTSYWFPY

SEQ ID NO: 10 - protein, 11 aa, synthetic construct
KASQDINSYLS

SEQ ID NO: 11 - protein, 7 aa, synthetic construct
RANRLVD

SEQ ID NO: 12 - protein, 9 aa, synthetic construct
LQFDEFPYT

SEQ ID NO: 13 - protein, 7 aa, synthetic construct
GYTFSSN

SEQ ID NO: 14 - protein, 6 aa, synthetic construct
LPGSGS

SEQ ID NO: 15 - protein, 8 aa, synthetic construct
EELGYFDY

SEQ ID NO: 16 - protein, 11 aa, synthetic construct
RASQDINNYLN

SEQ ID NO: 17 - protein, 7 aa, synthetic construct
YTSRLHS

SEQ ID NO: 18 - protein, 9 aa, synthetic construct
QQGSTLPPT

SEQ ID NO: 19 - protein, 103 aa, synthetic construct
EVQLQQPGAELVKPGGSVKLSCKASGYSITSDYWVKQRPGRQLEWIGSYIGSKISLTVDKPSSTAYMQLSSLTSEDSAVYYCARTIRNYFDYWGQGTTVTVSS

SEQ ID NO: 20 - protein, 108 aa, synthetic construct
DIVMTQTHKFMSTSVGDRVSLTCSASQGISNYLNWYQEKPGQSPKLLIYSDTSSLHSGVPARFTGTQSGTDFTFTISSVQAEDEALYFCQQYSNFPCTFGAGTKLELK

SEQ ID NO: 21 - protein, 104 aa, synthetic construct
EVQLQQPGAELVKPGGSVKLSCKASGFNIKDYWVKQRPGRQLEWIGDPENGNKISLTVDKPSSTAYMQLSSLTSEDSAVYYCARYGTSYWFPYWGQGTTVTVSS

SEQ ID NO: 22 - protein, 108 aa, synthetic construct
DIVMTQTHKFMSTSVGDRVSLTCKASQDINSYLSWYQEKPGQSPKLLIYSRANRLVDGVPARFTGTQSGTDFTFTISSVQAEDEALYFCLQFDEFPYTFGAGTKLELK

SEQ ID NO: 23 - protein, 103 aa, synthetic construct
EVQLQQPGAELVKPGGSVKLSCKASGYTFSSNWVKQRPGRQLEWIGLPGSGSKISLTVDKPSSTAYMQLSSLTSEDSAVYYCAREELGYFDYWGQGTTVTVSS

SEQ ID NO: 24 - protein, 108 aa, synthetic construct
DIVMTQTHKFMSTSVGDRVSLTCRASQDINNYLNWYQEKPGQSPKLLIYSYTSRLHSGVPARFTGTQSGTDFTFTISSVQAEDEALYFCQQGSTLPPTFGAGTKLELK

SEQ ID NO: 25 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 106 aa, synthetic construct
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 574 aa, Respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKRSCRISNIETVIEFQHKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIEIIVILLSLIAVGLLLYCKARSTPVTLSKDQLSGINNIAFSN

SEQ ID NO: 28 - protein, 108 aa, synthetic construct
DIVMTQTHKFMSTSVGDRVSLTCSASQGISNYLNWYQEKPGQSPKLLIYSDTSSLHSGVPARFTGTQSGTDFTFTISSVQAEDEALYFCQQYSNFPFTFGAGTKLELK

(19) United States
(12) Patent Application Publication
(10) Pub. No.: US 2026/0116955 A1
(43) Pub. Date: Apr. 30, 2026

(54) ANTI-RESPIRATORY SYNCYTIAL VIRUS ANTIBODY AND RELATED USE THEREOF

(71) Applicant: HEAVYBIO, INC, Shenzhen, Guangdong (CN)

(72) Inventors: Yujia LIN, Shenzhen, Guangdong (CN); Chunxi GONG, Shenzhen, Guangdong (CN); Haiqiao OUYANG, Shenzhen, Guangdong (CN); Honghua QUAN, Shenzhen, Guangdong (CN); Yanfei LI, Shenzhen, Guangdong (CN); Yunbo WU, Shenzhen, Guangdong (CN); Langshan CHI, Shenzhen, Guangdong (CN); Xuesheng WU, Shenzhen, Guangdong (CN); Meixiang YOU, Shenzhen, Guangdong (CN)

(21) Appl. No.: 19/115,516

(22) PCT Filed: Sep. 15, 2023
(86) PCT No.: PCT/CN2023/118965

§ 371 Date: Mar. 26, 2025

(30) Foreign Application Priority Data
Sep. 27, 2022 (CN) 202211182828.4
Sep. 27, 2022 (CN) 202211182850.9
Sep. 27, 2022 (CN) 202211196049.X

(51) Int. Cl. C07K 16/11; C12N 15/85; G01N 33/569

(52) U.S. Cl. CPC C07K 16/11; C12N 15/85; G01N 33/56983; C07K 2317/14; C07K 2317/565; C12N 2800/107; G01N 2333/135; G01N 2800/26

(57) ABSTRACT

The present disclosure relates to a novel anti-respiratory syncytial virus (RSV) monoclonal antibody, a method for preparing the antibody, and related applications thereof for immunoassay. The anti-RSV monoclonal antibody of the present disclosure exhibits high sensitivity and high specificity against RSV, and thus can be used for RSV detection at a nanoscale concentration on a chromatographic platform, and achieve more sensitive detection at a picoscale concentration in other platforms such as those for chemiluminescent assays.

### TECHNICAL FIELD

The present disclosure relates to the field of virus detection, and specifically relates to an anti-respiratory syncytial virus (RSV) monoclonal antibody, its preparation method and its related applications in immunodetection.

### BACKGROUND

Respiratory syncytial virus (RSV) belongs to the genus Pneumovirus in the family Paramyxoviridae, and is an enveloped, negative-strand RNA virus. The RSV virus infects an estimated 64 million people worldwide each year, resulting in hospitalization of more than 3 million infants and young children, and 160,000 deaths. Therefore, sensitive and rapid detection of RSV infection is extremely important, and antibodies against RSV virus play an important role in immune diagnosis of RSV virus infection.

RSV expresses two main surface glycoproteins: fusion protein (F protein) and attachment protein (G protein). The F protein exhibits less genetic variability than the G protein, and binds to the specific receptor on the membrane surface of the host cell to mediate the fusion of the viral envelope with the plasma membrane of the host cell, causing the viral nucleic acid to enter the host cell and be replicated therein. Therefore, the RSV F protein is a very attractive target for both immunodetection and antibody-based therapeutics.

At present, the currently available antibodies against RSV F protein are deficient in sensitivity and specificity, clinically cause false negative results, and have much room for improvement. Therefore, there is still a need in the art for an anti-RSV F protein antibody with high specificity and sensitivity.

### SUMMARY

As mentioned above, there is a need for an anti-RSV antibody with high specificity and high sensitivity in the field.

In the present disclosure, the purified RSV fusion protein (F protein) was used as an immunogen to induce an immune response in mice, then the hybridoma techniques were used to produce hybridoma cell strains capable of secreting anti-RSV monoclonal antibodies, the hybridoma cells were subject to in vitro roller bottle culture, a large number of anti-RSV monoclonal antibodies were obtained by harvesting the supernatant, and anti-RSV antibodies with high sensitivity and high specificity were obtained via screening. To this end, the present invention is realized.

In a first aspect, the present disclosure provides an anti-respiratory syncytial virus (RSV) monoclonal antibody or an antigen-binding fragment thereof, comprising a heavy-chain variable region and a light-chain variable region, wherein:
1) the heavy-chain variable region comprises heavy-chain complementarity-determining regions V_{H} CDR1, V_{H} CDR2 and V_{H} CDR3 with amino acid sequences shown in SEQ ID NO: 1-3, respectively, and the light-chain variable region comprises light-chain complementarity-determining regions V_{L} CDR1, V_{L} CDR2 and V_{L} CDR3 with amino acid sequences shown in SEQ ID NO: 4-6, respectively, wherein the sequence shown in SEQ ID NO: 6 is QQYSNFPXT, wherein X is C or F;
2) the heavy-chain variable region comprises heavy-chain complementarity-determining regions V_{H} CDR1, V_{H} CDR2 and V_{H} CDR3 with amino acid sequences shown in SEQ ID NO: 7-9, respectively, and the light-chain variable region comprises light-chain complementarity-determining regions V_{L} CDR1, V_{L} CDR2 and V_{L} CDR3 with amino acid sequences shown in SEQ ID NO: 10-12, respectively; or
3) the heavy-chain variable region comprises heavy-chain complementarity-determining regions V_{H} CDR1, V_{H} CDR2 and V_{H} CDR3 with amino acid sequences shown in SEQ ID NO: 13-15, respectively, and the light-chain variable region comprises light-chain complementarity-determining regions V_{L} CDR1, V_{L} CDR2 and V_{L} CDR3 with amino acid sequences shown in SEQ ID NO: 16-18, respectively.

In a second aspect, the present disclosure provides a hybridoma cell deposited under accession number CGMCC No. 45238, CGMCC No. 45239 or CGMCC No. 45240 in the China General Microbiological Culture Collection Center (CGMCC), Institute of Microbiology, Chinese Academy of Sciences, located at Room 3, NO. 1 West Beichen Road, Chaoyang District, Beijing, China, on Jul. 14, 2022.

In a third aspect, the present disclosure provides a nucleic acid molecule encoding the anti-RSV monoclonal antibody or the antigen-binding fragment thereof according to the first aspect.

In a fourth aspect, the present disclosure provides a vector comprising the nucleic acid molecule according to the third aspect.

In a fifth aspect, the present disclosure provides an expression cell comprising the vector according to the fourth aspect.

In a sixth aspect, the present disclosure provides use of the anti-RSV monoclonal antibody or the antigen-binding fragment thereof according to the first aspect for detecting respiratory syncytial virus (RSV).

In a seventh aspect, the present disclosure provides a method for detecting respiratory syncytial virus (RSV), comprising a step of using the anti-RSV monoclonal antibody or the antigen-binding fragment thereof according to the first aspect.

In an eighth aspect, the present disclosure provides a method for diagnosing respiratory syncytial virus (RSV) infection, comprising a step of testing a sample from a subject with the anti-RSV monoclonal antibody or the antigen-binding fragment thereof according to the first aspect.

In a ninth aspect, the present disclosure provides a kit for detecting respiratory syncytial virus (RSV), comprising at least one anti-RSV monoclonal antibody or the antigen-binding fragment thereof according to the first aspect, and instructions for use to guide the detection of respiratory syncytial virus (RSV).

The present disclosure provides a novel anti-RSV monoclonal antibody which exhibits high sensitivity and high specificity against RSV, and thus can be used to detect RSV at a nanoscale or even picoscale concentration, thereby greatly improving sensitivity of RSV detection and analysis. The anti-RSV antibody of the present disclosure can be applied to various immunochromatographic assays, such as fluorescent microsphere chromatographic assays; colloidal gold chromatographic assays; colored microsphere chromatographic assays; ELISA assays in direct, indirect, sandwich and competitive formats; chemiluminescent assays; electrochemiluminescent assays, etc. The anti-RSV antibody of the present disclosure exhibits high sensitivity and high specificity against RSV, and thus can be used for detecting RSV at a nanoscale concentration on a chromatographic platform, and achieve more sensitive detection at a picoscale concentration in other platforms such as those for chemiluminescent assays. Therefore, the anti-RSV monoclonal antibody of the present disclosure provides a rapid, efficient, accurate and cost-effective solution for clinical detection and diagnosis of respiratory syncytial virus.

### BRIEF DESCRIPTION OF THE DRAWINGS

In order to more clearly illustrate the particular embodiments of the present disclosure or the technical solutions in the prior art, the drawings, to which the particular embodiments or the description of the prior art refers, will be briefly described in the following.

FIG. 1 shows the SDS-PAGE electrophersis image of antibodies RSV101, RSV200 and RSV300 of the present disclosure (5 μg loaded per lane);

FIG. 2 shows OD₄₅₀ₙₘ absorbance curves of antibodies RSV101, RSV200 and RSV300 of the present disclosure measured on a microplate reader;

FIG. 3 shows the OD₄₅₀ₙₘ absorbance curves of antibodies RSV101 and RSV102 of the present disclosure measured on a microplate reader;

FIG. 4 shows a schematic diagram of the colloidal gold color chart used in the present disclosure; and

FIG. 5 shows the sensitivity curve of an exemplary fluorescent microsphere immunochromatographic test strip prepared from the antibodies of the present disclosure, against RSV fusion protein recombinant antigen.

### DETAILED DESCRIPTION OF THE EMBODIMENTS

Hereinafter, the present disclosure will be described in detail in conjunction with the accompanying drawings. It should be understood that the following description only illustrates the present disclosure by way of example and is not intended to limit the scope of the present disclosure, and the protection scope of the present disclosure is subject to the appended claims. Furthermore, it will be appreciated by those skilled in the art that the technical solutions of the present disclosure may be modified without departing from the spirit and scope of the present disclosure. If not specifically indicated, the technical means used in the embodiments are conventional means well known to those skilled in the art.

Unless otherwise defined, all technical and scientific terms used herein have the same meaning as commonly understood by one of ordinary skill in the art to which the subject matter described herein belongs. Before the present disclosure is described in detail, the following definitions are provided for a better understanding of the present disclosure.

In the case of providing a numerical range, such as a concentration range, a percentage range or a ratio range, it should be understood that, unless the context clearly specifies otherwise, individual intermediate values to one-tenth of the lower limit unit between the upper and lower limits of the range, and any other described values or intermediate values in the described range are included in the described subject matter. The upper and lower limits of these smaller ranges can be included in the described smaller range independently, and such embodiments are also included in the described subject matter, subject to any specific excluded limit value in the described range. In the case where the described range includes one or two limit values, the range excluding any one or two of those included limit values is also included in the described subject matter.

In the context of the present disclosure, many embodiments are described by using the expression “comprising”, “containing” or “consisting essentially/substantially of” and their grammatical variants. The expression “comprising”, “containing” or “consisting essentially/substantially of” or their grammatical variants is generally understood as an open-ended expression, indicating that, in addition to the elements, components, assemblies, method steps, etc. specifically those listed after the expression, other elements, components, assemblies, method steps, etc. are also included. In addition, the expression “comprising”, “containing” or “consisting essentially/substantially of” herein can also be understood as a closed-ended expression in some cases, indicating that only the elements, components, assemblies, method steps specifically listed after the expression are included, and no other elements, components, assemblies, method steps are included. In such case, the expression is equivalent to the expression “consisting of”.

In order to better understand the present teachings without limiting the scope of the present teachings, unless otherwise indicated, all numbers indicating quantities, percentages or ratios, and other numerical values, used in the description and claims shall be construed as being modified by the term “about” or “approximately” in all cases. Therefore, unless indicated to the contrary, the numerical parameters set forth in the following description and the appended claims are approximations, which may vary depending on the desired properties sought to be obtained. At a minimum, each numerical parameter should at least be interpreted in light of the numerical values of reported valid numbers and by applying ordinary rounding techniques.

As used herein, the term “antibody” refers to an immunoglobulin molecule typically composed of two pairs of polypeptide chains, with each pair having one “light” (L) chain and one “heavy” (H) chain. The antibody light chain can be classified as kappa (κ) and lambda (λ) light chains. The heavy chain can be classified as μ, δ, γ, α, or ε, and accordingly the antibody isotypes can be defined as IgM, IgD, IgG, IgA, and IgE, respectively. Within the light and heavy chains, the variable and constant regions are connected by a “J” region (hinge region) of about 12 or more amino acids, and the heavy chain further contains a “D” region of about 3 or more amino acids. Each heavy chain consists of a heavy-chain variable region (V_{H}) and a heavy-chain constant region (C_{H}). The heavy-chain constant region consists of three domains (C_{H1}, C_{H2}, and C_{H3}). Each light chain consists of a light-chain variable region (V_{L}) and a light-chain constant region (C_{L}). The light-chain constant region consists of one domain C_{L}. The constant regions of the antibody mediate the binding of immunoglobulin to host tissue(s) or factor(s), including various cells (e.g., effector cells) of the immune system and the first component (C1q) of the classical complement system. The V_{H} and V_{L} regions can be further subdivided into regions of hypervariability, termed as complementarity-determining regions (CDRs), interspersed with regions that are more conserved, termed as framework regions (FRs). For each heavy chain or light chain, its variable region each contains three CDRs, namely CDR1, CDR2 and CDR3. Therefore, each V_{H} or V_{L} comprises three CDRs and four FRs arranged from the amino terminus to the carboxyl terminus in the following order: FR1, CDR1, FR2, CDR2, FR3, CDR3, FR4. The variable regions (V_{H} and V_{L}) of each heavy chain/light chain pair form an antigen-binding site, respectively.

The rules for assigning amino acids to the above regions or domains are defined in several literatures: Kabat Sequences of Proteins of Immunological Interest (National Institutes of Health, Bethesda M.d. (1987 and 1991)); Chothia &Lesk J. Mol. Biol. 1987; 196:901-917; Chothia et al., Nature 1989; 342:878-883; Ehrenmann, Francois, Quentin Kaas, and Marie-PauleLefranc. “IMGT/3Dstructure-DB and IMGT/DomainGapAlign: a database and a tool for immunoglobulins or antibodies, T cell receptors, MHC, IgSF and MhcSF.” Nucleic acids research 2009; 38(suppl_1): D301-D307.

The exact boundaries of the CDRs have been defined differentially according to different systems. The Kabat system not only provides a well-defined residue numbering system applicable to any variable region of an antibody, but also provides precise residue boundaries that define the three CDRs, which are referred to as Kabat CDRs. Chothia discovered that certain sub-regions within the Kabat CDRs share almost identical conformations of peptide backbone, despite their great diversity at the amino acid sequence level. These sub-regions are called as Chothia CDRs, which have boundaries overlapping with those of the Kabat CDRs. The above overlapping boundaries were again described by Padlan and MacCallum. The CDR boundary definitions may not strictly adhere to the above system, such as the AbM definition. In this disclosure, CDRs can be defined according to any of these systems, although preferred embodiments of the present disclosure use the antibody numbering system of Chothia et al. to define CDRs. According to the Chothia numbering system, an antibody has a V_{H} CDR1 located at positions 26 to 32, a V_{H} CDR2 located at positions 52 to 57, a V_{H} CDR3 located at positions 99 to 108, a V_{L} CDR1 located at positions 24 to 39, a V_{L} CDR2 located at positions 55 to 61, and a V_{L} CDR3 located at positions 94 to 102.

As used herein, the term “monoclonal antibody” or “mAb” refers to an antibody or an antibody fragment from a population of highly homologous antibody molecules, that is, a population of completely identical antibody molecules with the exception of naturally occurring spontaneous mutations that may arise. The antibody molecule is an immunoglobulin, either naturally occurring or obtained partially or completely by synthetic methods. The antibody molecules also include all polypeptides or proteins having an antigen-binding domain. Antibody fragments having an antigen-binding domain are molecules such as Fabs, scFvs, Fvs, dAbs, Fds, and bifunctional antibodies. Monoclonal antibodies have high specificity for a single epitope on an antigen. As opposed to monoclonal antibodies, polyclonal antibodies usually contain at least two or more different antibodies which recognize different epitopes on an antigen. Monoclonal antibodies are generally produced using the hybridoma technique first reported by Kohler et al. (Köhler G, Milstein C. Continuous cultures of fused cells secreting antibody of predefined specificity [J]. nature, 1975; 256(5517): 495), but they can also be obtained by recombinant DNA techniques (see, e.g., U.S. Pat. No. 4,816,567). As used herein, the terms “monoclonal antibody” and “mAb” have the same meaning and are used interchangeably; the terms “polyclonal antibody” and “pAb” have the same meaning and are used interchangeably; and the terms “polypeptide” and “protein” have the same meaning and are used interchangeably. And in the present disclosure, amino acids are usually represented by single-letter and three-letter abbreviations known in the art. For example, alanine can be represented by Ala or A, glycine can be represented by Gly or G, valine can be represented by Val or V, leucine can be represented by Leu or L, isoleucine can be represented by Ile or I, proline can be represented by Pro or P, phenylalanine can be represented by Phe or F, tyrosine can be represented by Tyr or Y, tryptophan can be represented by Trp or W, serine can be represented by Ser or S, threonine can be represented by Thr or T, cysteine can be represented by Cys or C, methionine can be represented by Met or M, asparagine can be represented by Asn or N, glutamine can be represented by Gln or Q, aspartic acid can be represented by Asp or D, glutamic acid can be represented by Glu or E, lysine can be represented by Lys or K, arginine can be represented by Arg or R, and histidine can be represented by His or H.

As used herein, the term “recombinant antibody” refers to an antibody obtained by cloning an antibody gene into an expression vector and then transfecting the expression vector into a suitable host cell line for expression through molecular biological techniques. The recombinant antibody-encoding gene may be identical to or different from the antibody-encoding gene of natural source. For example, the complete coding gene of an antibody obtained by immunizing an animal can be cloned into an expression vector for expression, thereby obtaining an antibody that is completely identical to the antibody obtained by immunizing the animal, or alternatively the gene encoding the variable regions (including the heavy-chain variable region and the light-chain variable region) of an antibody obtained by immunizing an animal can be cloned into an expression vector, together with the gene encoding the constant regions of an antibody from another species (such as human) for expression, thereby obtaining an antibody comprising the heavy-chain and light-chain variable region sequences from one species and the constant region sequences from another species, such as an antibody having mouse heavy-chain and light-chain variable regions connected to the human constant regions. Such an antibody is generally referred to as a “chimeric antibody” in the art.

As used herein, the term “antigen-binding fragment” refers to a fragment or antibody analog which is derived from an antibody and is capable of binding to an antigen, and generally comprises at least a part of the antigen-binding region or variable region (e.g., one or more CDRs) of the parental antibody. The antigen-binding fragment retains at least some of the binding activity of the parental antibody. Typically, the antigen-binding fragment retains at least 10% of the binding activity of the parental antibody, where the activity is expressed on a molar basis. Specifically, the antigen-binding fragment retains at least 20%, 50%, 70%, 80%, 90%, 95% or 100% or more of the binding affinity of the parental antibody to the target. Examples of the antigen-binding fragments include, but are not limited to, Fab, Fab′, F(ab′)₂ or Fv fragments, linear antibodies, single-chain antibodies, nanobodies, domain antibodies, and multispecific antibodies. The “Fab fragment” is composed of one light chain, and the C_{H1} domain and the variable region of one heavy chain. The heavy chain of the Fab molecule cannot form any disulfide bond with another heavy chain molecule. The “Fab′ fragment” contains one light chain, and a portion of one heavy chain (including the V_{H} domain, the C_{H1} domain, and the region between the C_{H1} and C_{H2} domains); thus, two Fab′ fragments can form an F(ab′)₂ molecule via interchain disulfide bonds between their two heavy chains. An “Fv region” contains the variable regions from both the heavy and light chains, but lacks the constant region.

As used herein, the term “specific binding” refers to a non-random binding reaction between two molecules, such as the reaction between an antibody and its antigen.

In the present disclosure, the antibody-encoding nucleotide sequence is also amplified via PCR by using primers. In the primer sequence, some positions only involve a single base, such as any one of adenine (A), guanine (G), cytosine (C) and thymine (T), while some positions involve a combination of two, three or four bases. In the latter case, these bases are called as degenerate bases of each other, and are mainly determined based on codon degeneracy. Degenerate bases are represented by letters R, Y, M, K, S, W, H, B, V, D, and N, where R represents A/G, Y represents C/T, M represents A/C, K represents G/T, S represents C/G, W represents A/T, H represents A/T/C, B represents G/T/C, V represents G/A/C, D represents G/A/T, and N represents A/T/C/G.

The terms sequence “identity” or “homology” as used herein have well-established meanings in the art. The percentage of sequence identity between two nucleic acid or polypeptide molecules or regions can be calculated using publicly available methods. The sequence identity can be determined along the entire length of a polynucleotide or polypeptide or along a region of the molecule (See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Though there are many methods for determining identity between two polynucleotides or polypeptides, it is well known to those of skill in the art that the term “identity” is applicable to conservative amino acid substitutions in peptides or proteins, and the conservative amino acid substitutions can be conventionally made without altering the biological activity of the resulting molecule. Generally, those skilled in the art appreciate that single amino acid substitutions in non-essential regions of a polypeptide do not substantially alter the biological activity (see, e.g., Watson et al., Molecular Biology of the Gene, 4th Edition, 1987, The Benjamin/Cummings Pub. co., p. 224).

As mentioned above, the present disclosure aims to provide an anti-RSV monoclonal antibody with high sensitivity and high specificity.

In a first aspect, the present disclosure provides an anti-respiratory syncytial virus (RSV) monoclonal antibody or an antigen-binding fragment thereof, comprising a heavy-chain variable region and a light-chain variable region, wherein:
i. the heavy-chain variable region comprises heavy-chain complementarity-determining regions V_{H} CDR1, V_{H} CDR2 and V_{H} CDR3 with amino acid sequences shown in SEQ ID NO: 1-3, respectively, and the light-chain variable region comprises light-chain complementarity-determining regions V_{L} CDR1, V_{L} CDR2 and V_{L} CDR3 with amino acid sequences shown in SEQ ID NO: 4-6, respectively, wherein the sequence shown in SEQ ID NO: 6 is QQYSNFPXT, wherein X is C or F;
ii. the heavy-chain variable region comprises heavy-chain complementarity-determining regions V_{H} CDR1, V_{H} CDR2 and V_{H} CDR3 with amino acid sequences shown in SEQ ID NO: 7-9, respectively, and the light-chain variable region comprises light-chain complementarity-determining regions V_{L} CDR1, V_{L} CDR2 and V_{L} CDR3 with amino acid sequences shown in SEQ ID NO: 10-12, respectively; or
iii. the heavy-chain variable region comprises heavy-chain complementarity-determining regions V_{H} CDR1, V_{H} CDR2 and V_{H} CDR3 with amino acid sequences shown in SEQ ID NO: 13-15, respectively, and the light-chain variable region comprises light-chain complementarity-determining regions V_{L} CDR1, V_{L} CDR2 and V_{L} CDR3 with amino acid sequences shown in SEQ ID NO: 16-18, respectively.

In a preferred embodiment, the heavy-chain variable region comprises heavy-chain complementarity-determining regions V_{H} CDR1, V_{H} CDR2 and V_{H} CDR3 with amino acid sequences shown in SEQ ID NO: 1-3, respectively, and the light-chain variable region comprises light-chain complementarity-determining regions V_{L} CDR1, V_{L} CDR2 and V_{L} CDR3 with amino acid sequences shown in SEQ ID NO: 4-6, respectively, wherein the sequence shown in SEQ ID NO: 6 is QQYSNFPXT, wherein X is F.

In a particular embodiment, the antibody may be a full-length antibody comprising variable regions and constant regions. In the antibody of the present disclosure, any framework regions (FRs) and any constant regions may be used. The FRs or constant regions used in the antibody of the present disclosure may have amino acid sequences identical to those of original FRs or constant regions as source, or have different amino acid sequences obtained by modifying the amino acid sequences of original FRs or constant regions via substitution, deletion, addition and/or insertion of one or more amino acids. The structural elements supporting the CDRs or CDR sets of the present disclosure typically reside in heavy or light chain sequences of the antibody or major portions thereof, wherein the CDRs or CDR sets are located at positions corresponding to the CDRs or CDR sets of naturally occurring V_{H} and V_{L} antibody variable domains encoded by rearranged immunoglobulin genes.

As an example, the framework regions (FRs) may have the following sequence:

HFR1 (heavy-chain framework region 1):
QLQQPGAELVRPGASVKLSCKAS
HFR2 (heavy-chain framework region 2):
WVKQRPGQGLEWIG
HFR3 (heavy-chain framework region 3):
KATLTVDTSSSTAYMQLSSLTSEDSAVYFC AR
HFR4 (heavy-chain framework region 4):
WGQGTLVTVSS
LFR1 (light-chain framework region 1):
DIVLTQSPASLSVATGEKVTIRC
LFR2 (light-chain framework region 2):
WYQQKPGQPPKLLIY
LFR3 (light-chain framework region 3):
GVPDRFSGSGSGTDFTLKISPMEEDDTAMY FC
LFR4 (light-chain framework region 4):
FGGGTKLEI
As another example, the framework regions (FRs)
may alternatively have the following sequence:
HFR1 (heavy-chain framework region 1):
QLQQPGAELVKPGGSVKLSCKAS
HFR2 (heavy-chain framework region 2):
WVKQRPGRQLEWIG
HFR3 (heavy-chain framework region 3):
KISLTVDKPSSTAYMQLSSLTSEDSAVYYC AR
HFR4 (heavy-chain framework region 4):
WGQGTTVTVSS
LFR1 (light-chain framework region 1):
DIVMTQTHKFMSTSVGDRVSLTC
LFR2 (light-chain framework region 2):
WYQEKPGQSPKLLIYS
LFR3 (light-chain framework region 3):
GVPARFTGTQSGTDFTFTISSVQAEDEALY FC
LFR4 (light-chain framework region 4):
FGAGTKLELK

In a particular embodiment, the heavy-chain variable region comprises an amino acid sequence shown in SEQ ID NO: 19, or a sequence having at least 80%, at least 85%, at least 90% or at least 95% identity with the amino acid sequence shown in SEQ ID NO: 19, and the light-chain variable region comprises an amino acid sequence shown in SEQ ID NO: 20, or a sequence having at least 80%, at least 85%, at least 90% or at least 95%, or even at least 99%, 99.1%, 99.2%, 99.3%, 99.4%, 99.5%, 99.6%, 99.7%, 99.8%, or 99.9% identity with the amino acid sequence shown in SEQ ID NO: 20.

In a particular embodiment, the heavy-chain variable region comprises an amino acid sequence shown in SEQ ID NO: 19, or a sequence having at least 80%, at least 85%, at least 90% or at least 95% identity with the amino acid sequence shown in SEQ ID NO: 19, and the light-chain variable region comprises an amino acid sequence shown in SEQ ID NO: 28, or a sequence having at least 80%, at least 85%, at least 90% or at least 95%, or even at least 99%, 99.1%, 99.2%, 99.3%, 99.4%, 99.5%, 99.6%, 99.7%, 99.8%, or 99.9% identity with the amino acid sequence shown in SEQ ID NO: 28.

In yet another particular embodiment, the heavy-chain variable region comprises an amino acid sequence shown in SEQ ID NO: 21, or a sequence having at least 80%, at least 85%, at least 90% or at least 95% identity with the amino acid sequence shown in SEQ ID NO: 21, and the light-chain variable region comprises an amino acid sequence shown in SEQ ID NO: 22, or a sequence having at least 80%, at least 85%, at least 90% or at least 95%, or even at least 99%, 99.1%, 99.2%, 99.3%, 99.4%, 99.5%, 99.6%, 99.7%, 99.8%, or 99.9% identity with the amino acid sequence shown in SEQ ID NO: 22.

In yet another particular embodiment, the heavy-chain variable region comprises an amino acid sequence shown in SEQ ID NO: 23, or a sequence having at least 80%, at least 85%, at least 90% or at least 95% identity with the amino acid sequence shown in SEQ ID NO: 23, and the light-chain variable region comprises an amino acid sequence shown in SEQ ID NO: 24, or a sequence having at least 80%, at least 85%, at least 90% or at least 95%, or even at least 99%, 99.1%, 99.2%, 99.3%, 99.4%, 99.5%, 99.6%, 99.7%, 99.8%, or 99.9% identity with the amino acid sequence shown in SEQ ID NO: 24.

In a particular embodiment, the antibody further comprises a constant region sequence, such as but not limited to a constant region sequence derived from any one of IgG, IgA, IgM, IgE and IgD. The constant region sequence may be selected based on actual needs by those skilled in the art, and is not specifically limited herein.

In yet another particular embodiment, the constant region sequence may be sourced from rats, mice, rabbits, goats, sheep, horses, dogs, cows, pigs, chickens, ducks, geese or humans, but is not limited thereto.

In a particular embodiment, the constant region of the antibody of the present disclosure is sourced from mice.

In some embodiments, the constant region sequence is sourced from mice.

In an exemplary embodiment, the constant region sequence may comprise or consist of the following sequence:

C_{H} with the amino acid sequence shown in SEQ ID
NO: 25:
AKTTAPSVYPLAPVCGDTTGSSVTLGCLVKGYFPEPVTLTWNSGSLSSG
VHTFPAVLQSDLYTLSSSVTVTSSTWPSQSITCNVAHPASSTKVDKKIE
PRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDW
MSGKEFKCKVNNKDLPAPIERTISKPKGSVRAPQVYVLPPPEEEMTKKQ
VTLTCMVTDFMPEDIYVEWTNNGKTELNYKNTEPVLDSDGSYFMYSKLR
VEKKNWVERNSYSCSVVHEGLHNHHTTKSFSRTPGK;
C_{L} with the amino acid sequence shown in SEQ ID
NO: 25:
ADAAPTVSIFPPSSEQLTSGGASVVCFLNNFYPKDINVKWKIDGSERQN
GVLNSWTDQDSKDSTYSMSSTLTLTKDEYERHNSYTCEATHKTSTSPIV
KSFNRNEC.

In a second aspect, the present disclosure provides a hybridoma cell deposited under accession number CGMCC No. 45238, CGMCC No. 45239 or CGMCC No. 45240 in the China General Microbiological Culture Collection Center (CGMCC), Institute of Microbiology, Chinese Academy of Sciences, located at Room 3, NO. 1 West Beichen Road, Chaoyang District, Beijing, China, on Jul. 14, 2022.

In a third aspect, the present disclosure provides a nucleic acid molecule encoding the anti-RSV monoclonal antibody or the antigen-binding fragment thereof according to the first aspect.

For those skilled in the art, where the amino acid sequence of a protein such as the anti-RSV monoclonal antibody of the present disclosure is known, it is within their capabilities to determine its nucleic acid coding sequence. In addition, in order to obtain a monoclonal antibody by recombinant means, the nucleic acid molecule can be cloned into a vector, and then the vector can be further introduced into an expression cell to express the antibody protein therein.

In a fourth aspect, the present disclosure provides a vector comprising the nucleic acid molecule according to the third aspect.

In a particular embodiment, the vector may be a plasmid vector, such as any one of pEE12, pCAGGS, pTOPO, pcDNA, pTT, pTT3, pEFBOS, pBV, pJV and pBJ.

In yet another particular embodiment, the vector may be a eukaryotic expression vector.

In a preferred embodiment, the pcDNA vector may be pCDNA3.1.

In a fifth aspect, the present disclosure provides an expression cell comprising the vector according to the fourth aspect.

The expression cell is prepared by introducing the above-mentioned nucleic acid molecule or vector into a host cell by molecular biological methods well known to those skilled in the art.

As mentioned above, the purified RSV fusion protein (F protein, SEQ ID NO: 27) was used as an immunogen to induce an immune response in mice, then the hybridoma techniques were used to produce hybridoma cell strains capable of secreting anti-RSV monoclonal antibodies, the hybridoma cells were subjected to in vitro roller bottle culture, a large number of anti-RSV antibodies were obtained by harvesting the supernatant, and anti-RSV antibodies with high sensitivity and specificity were obtained via screening, which were named as RSV101, RSV200 and RSV300.

The above-mentioned monoclonal antibodies and recombinant DNA techniques can be used to produce other antibodies or chimeric molecules that retain the specificity of the original antibodies. These techniques may include introducing DNA encoding variable regions or complementarity-determining regions (CDRs) of an antibody immunoglobulin into a eukaryotic expression vector containing DNA encoding constant regions or constant regions plus framework regions of a different immunoglobulin, or alternatively introducing both of the DNAs into a suitable eukaryotic expression vector, and then introducing the eukaryotic expression vector into an expression cell, such as a CHO host cell, to obtain a recombinant anti-RSV antibody, such as RSV101′, RSV200′ or RSV300′. On this basis, the inventors further mutated the light chain CDR3 of RSV101 from QQYSNFPCT to QQYSNFPFT, constructed an eukaryotic expression vector for recombinant antibody expression, introduced the vector into a CHO host cell, and screened out a cell strain stably-expressing the recombinant antibody, which was named as RSV102.

In an embodiment, the expression cell may be mammalian cells, such as Chinese hamster ovary cells, baby hamster kidney cells, monkey kidney cells, mouse thymoma cells, or human embryonic kidney cells. In a more particular embodiment, the expression cell may be, for example, SV40-transformed monkey kidney cells (COS-7, ATCC CRL1651), human embryonic kidney cells (HEK293 cells, or HEK293 cells subcloned for growth in suspension culture, Graham et al., 1977, J. Gen Virol.36: 59), baby hamster kidney cells (BHK, ATCC CCL10), Chinese hamster ovary cells/-DHFR1 (CHO, Urlaub et al., 1980, Proc. Natl. Acad. Sci. USA77:4216; e.g. DG44), mouse thymoma cells (NSO), mouse Sertoli cells (TM4, Mather, 1980, Biol. Reprod. 23:243-251), monkey kidney cells (CV-1, ATCC CCL70), African green monkey kidney cells (VERO-76, ATCC CRL-1587), human cervical cancer cells (HELA, ATCC CCL2), canine kidney cells (MDCK, ATCC CCL34), buffalo rat liver cells (BRL3A, ATCC CRL1442), human lung cells (W138, ATCC CCL75), human liver cells (HepG2, HB8065), mouse breast tumor cells (MMT060562, ATCC CCL51), TR1 cells (Mather et al., 1982, Annals N.Y.Acad.Sci.383:44-68), MRC5 cells, FS4 cells, etc., but not limited thereto.

In a preferred embodiment, the expression cell may be HEK293 cells.

In a sixth aspect, the present disclosure provides use of the anti-RSV monoclonal antibody or the antigen-binding fragment thereof according to the first aspect for detecting respiratory syncytial virus (RSV).

In a particular embodiment, detection of respiratory syncytial virus (RSV) in a sample (such as a blood or serum sample, a nasal swab sample, or a throat swab sample) from a subject can be used to diagnose whether the subject is infected with respiratory syncytial virus (RSV).

In a particular embodiment, the detection is performed by an immunochromatographic assay, an enzyme-linked antibody assay (ELISA), a chemiluminescent assay, or an electrochemiluminescent assay.

In a preferred embodiment, the enzyme-linked antibody assay (ELISA) may be in a direct, indirect, sandwich or competitive format.

In a preferred embodiment, the immunochromatographic assay includes but is not limited to fluorescent microsphere immunochromatographic assays, colloidal gold immunochromatographic assays, colored latex microsphere-based immunochromatographic assays, time-resolved fluorescent microsphere immunochromatographic assays, magnetic microsphere immunochromatographic assays or quantum dot immunochromatographic assays.

In the present disclosure, the anti-RSV monoclonal antibody may be used as a coating antibody. For example, the anti-RSV monoclonal antibody is bound to a solid phase such as a solid support. There is no particular limitation on the solid support used in the detection method of the present disclosure, and the solid support may be porous or non-porous material, such as magnetic beads, latex microspheres, fluorescent microspheres, microtiter plates, nitrocellulose membranes, microfluidic chips, etc. Without being bound by theory, the anti-RSV monoclonal antibody may also be used as a labeling antibody. For example, the anti-RSV monoclonal antibody is bound to magnetic beads, microspheres, enzymes, fluorescent dyes, biotin, streptavidin, quantum dots, colloidal gold, etc.

For example, when the colored latex microsphere-based immunochromatographic assay is performed, an immunochromatographic rapid test strip, which is obtained by assembling colored latex microspheres labeled with the anti-RSV antibody such as RSV101′ of the present disclosure, nitrocellulose membrane (NC membrane) coated with another anti-RSV antibody, such as RSV200′, a sample pad, an absorbent pad, and a polyester backing plate etc. in a conventional manner, may be used. During the assay, the analyte in a positive sample and the colored latex microspheres labeled with the anti-RSV antibody bind to each other and undergo an agglutination reaction at room temperature. Therefore, after a period of time, the results can be observed and interpreted visually.

For another example, when the colloidal gold immunochromatographic assay is performed, an anti-RSV antibody such as RSV102 may be used to label colloidal gold, another anti-RSV antibody such as RSV300′ of the present disclosure may be used to coat/dispense nitrocellulose membrane (NC membrane) to obtain the test line (T line), and these components can be assembled into a colloidal gold test strip according to the preparation method of the immune test strip. During the assay, the analyte in a positive sample and the colloidal gold-labeled anti-RSV antibody bind to each other and form a complex, and the complex subsequently binds to the coating antibody at the T line to form a sandwich complex, where the colloidal gold aggregates, precipitates and shows red, indicating that the sample is positive.

For another example, when the fluorescent microsphere immunochromatographic assay is performed, time-resolved fluorescent microspheres may be labeled with the anti-RSV monoclonal antibody such as RSV200′ of the present disclosure, and nitrocellulose membrane (NC membrane) may be coated with another anti-RSV antibody, such as RSV300′, and then these components, along with a sample pad etc., can be assembled into an immunochromatographic rapid test strip. During the assay, the analyte in a sample binds to the fluorescent microsphere-labeled antibody in the conjugate pad and migrates forward through capillary action. When reaching the test area, they further bind to another anti-RSV antibody immobilized at the test (T) line to form a dual-antibody sandwich complex. After the chromatographic assay is completed, the fluorescence intensity of the T line and the C line is read via an immunofluorescence analyzer and the T/C value is calculated. The content of the analyte in the sample can be calculated using the built-in standard curve in the analyzer.

It is worth noting that in the context of the present disclosure, the expression “another anti-RSV (monoclonal) antibody” refers to an antibody that can bind to the same antigen (preferably different epitope(s) of the same antigen) as the anti-RSV monoclonal antibody of the present disclosure, and may or may not be the anti-RSV monoclonal antibody of the present disclosure. For example, during the assay, when antibody RSV101′ is selected as the labeling antibody, RSV101′ can still be selected as the coating antibody, or RSV102, RSV200′ or RSV300′ can be selected as the coating antibody, or another different anti-RSV monoclonal antibody can be selected as the coating antibody.

Therefore, in the assay of the present disclosure, the labeling antibody and the coating antibody can be the same. That is to say, when the anti-RSV monoclonal antibody of the present disclosure is used for immunochromatographic assay to detect RSV virus, it may not only be used as a labeling antibody to label magnetic beads, microspheres, enzymes, fluorescent dyes, biotin, colloidal gold, etc., but also be used as a coating antibody to coat the solid support such as magnetic beads, latex microspheres, fluorescent microspheres, microtiter plates, nitrocellulose membranes, microfluidic chips, etc.

In a preferred embodiment, in the assay of the present disclosure, the labeling antibody may be different from the coating antibody. As an example, the inventors have found that when the anti-RSV monoclonal antibody RSV101′ or RSV102 of the present disclosure is used in combination with RSV300′, especially when RSV101′ is used in combination with RSV300′, no matter these antibodies are used as a labeling antibody or a coating antibody, they show significantly higher sensitivity in immunochromatographic reactions such as those based on colored latex microspheres or colloidal gold.

The assay of the present disclosure can be used in a point-of-care testing (POCT) or electrochemical immunoassay system. The assay according to the present disclosure or any exemplary forms thereof can be used in an automated and semi-automated system and optimized as appropriate.

In a seventh aspect, the present disclosure provides a method for detecting respiratory syncytial virus (RSV), comprising a step of using the anti-RSV monoclonal antibody or the antigen-binding fragment thereof according to the first aspect.

In an eighth aspect, the present disclosure provides a method for diagnosing respiratory syncytial virus (RSV) infection, comprising a step of testing a sample from a subject with the anti-RSV monoclonal antibody or the antigen-binding fragment thereof according to the first aspect.

In a particular embodiment, the sample may be a blood or serum sample, a throat swab sample or a stool sample, but is not limited thereto.

In a particular embodiment, the testing is performed by an immunochromatographic assay, an enzyme-linked antibody assay (ELISA), a chemiluminescent assay, or an electrochemiluminescent assay.

In a preferred embodiment, the enzyme-linked antibody assay (ELISA) may be in a direct, indirect, sandwich or competitive format.

In a preferred embodiment, the immunochromatographic assay includes but is not limited to fluorescent microsphere immunochromatographic assays, colloidal gold immunochromatographic assays, colored latex microsphere-based immunochromatographic assays, time-resolved fluorescent microsphere immunochromatographic assays, magnetic microsphere immunochromatographic assays or quantum dot immunochromatographic assays.

As mentioned above, the anti-RSV monoclonal antibody may be used as a coating antibody in detection. For example, the anti-RSV monoclonal antibody is bound to a solid phase such as a solid support. There is no particular restriction on the solid support used in the detection method of the present disclosure, and the solid support may be porous or non-porous material, such as magnetic beads, latex microspheres, fluorescent microspheres, microtiter plates, nitrocellulose membranes, microfluidic chips, etc. Similarly, the anti-RSV monoclonal antibody may also be used as a labeling antibody. For example, the anti-RSV monoclonal antibody is bound to magnetic beads, microspheres, enzymes, fluorescent dyes, biotin, streptavidin, quantum dots, colloidal gold, etc.

In a preferred embodiment, in the testing method of the present disclosure, the labeling antibody is different from the coating antibody. As an example, the inventors have found that when the anti-RSV monoclonal antibody RSV101′ or RSV102 of the present disclosure is used in combination with RSV300′, no matter these antibodies are used as a labeling antibody or a coating antibody, they show significantly higher sensitivity in immunochromatographic reactions such as those based on colored latex microspheres or colloidal gold.

In a ninth aspect, the present disclosure provides a kit for detecting respiratory syncytial virus (RSV), comprising at least one anti-RSV monoclonal antibody or the antigen-binding fragment thereof according to the first aspect, and instructions for use to guide the detection of respiratory syncytial virus (RSV).

In a particular embodiment, the kit may comprise at least two different anti-RSV monoclonal antibodies or antigen-binding fragments thereof, such as RSV101′ and RSV300′, to be used as the labeling antibody and the coating antibody in immunoassays, respectively.

In yet another particular embodiment, the kit may be used for clinical diagnosis of respiratory syncytial virus (RSV) infection.

The kit of the present disclosure may be used in a point-of-care testing (POCT) or electrochemical immunoassay system. The kit according to the present disclosure and any exemplary form thereof may be used in an automated and semi-automated system and optimized.

### EXAMPLE

In the following examples, the preparation method of the antibodies of the present disclosure and the characterization of related properties of the antibodies are illustrated. Unless otherwise specified, the experimental methods used are all conventional methods, and unless otherwise specified, the experimental materials used in the following examples were all purchased from commercial reagent suppliers. Unless otherwise defined, all technical and scientific terms used herein have the same meaning as those commonly understood by those skilled in the technical field of the present disclosure.

It should be noted that the terms used in the specification of the present disclosure are only for the purpose of describing specific examples, but not intended to limit the present disclosure. The above summary and the following detailed description are only for the purpose of specifically explaining the present disclosure, but not intended to limit the present disclosure in any way. Without departing from the spirit and scope of the present disclosure, the scope of the present disclosure is determined by the appended claims.

### Example 1: Preparation of Anti-RSV F Protein Monoclonal Antibodies

Immunization: The purified RSV F protein (SEQ ID NO: 27 MELPILKANAITTILAAVTFCFASSQNITEEFYQSTCSAVSKGYLSALRTGWYTSVITIELSNIKENKCNGTDAKVKL MKQELDKYKNAVTELQLLMQSTPAANNRARRELPRFMNYTLNNTKKTNVTLSKKRKRRFLGFLLGVGSAIASGI AVSKVLHLEGEVNKIKSALLSTNKAVVSLSNGVSVLTSKVLDLKNYIDKQLLPIVNKRSCRISNIETVIEFQHKNNRL LEITREFSVNAGVTTPVSTYMLTNSELLSLINDMPITNDQKKLMSNNVQIVRQQSYSIMSIIKEEVLAYVVQLPLY GVIDTPCWKLHTSPLCTTNTKEGSNICLTRTDRGWYCDNAGSVSFFPQAETCKVQSNRVFCDTMNSLTLPSEVN LCNVDIFNPKYDCKIMTSKTDVSSSVITSLGAIVSCYGKTKCTASNKNRGIIKTFSNGCDYVSNKGVDTVSVGNTLY YVNKQEGKSLYVKGEPIINFYDPLVFPSDEFDASISQVNEKINQSLAFIRKSDELLHNVNAGKSTTNIMITTIIIElIVIL LSLIAVGLLLYCKARSTPVTLSKDQLSGINNIAFSN) was used as an immunogen to immunize female BALB/c mice aged 6-8 weeks. The mice were immunized 4 times at 2-week intervals with a dose of 100 μg/mouse. For the first immunization, the RSV F protein was mixed with Freund's complete adjuvant (Sigma-Aldrich) in equal volumes and injected subcutaneously at multiple sites on the back of mice. For the subsequent three immunizations, the RSV F protein was mixed with Freund's incomplete adjuvant (Sigma-Aldrich) in equal volumes and injected intraperitoneally. Seven days after the fourth immunization, blood was collected from mice by tail-cutting, and serum was isolated from the blood, and tested for antibody titer by indirect ELISA, to observe the immune response. Mice with serum antibody titers higher than 1:10000 were selected for subsequent cell fusion. Three days prior to cell fusion, the mice were injected intraperitoneally with the RSV F protein without adjuvant for booster immunization (100 μg/mouse).

Construction of hybridoma cells: On the day of fusion, the spleens of the immunized mice were taken under sterile conditions and homogenized into single-cell suspensions. Murine myeloma cells (SP2/0) were combined with the spleen cells from the immunized BALB/c mice described above at a ratio of 1:5, mixed thoroughly, and washed twice before PEG fusion. Next, the cells were added with pre-warmed PEG1500, gently shaken, washed in pre-warmed serum-free RPMI-1640 medium, and then re-suspended in HAT selective medium. The cell suspension was plated into a 96-well plate at 200 μL/well, and the cells were cultured at 37° C. and 5% CO₂. After 4 to 7 days of culture, the cells were further cultured with HT medium. When the fused cells grew to cover 1/10-1/5 of the bottom area of the wells of the 96-well plate, the supernatant was collected for antibody detection.

Screening of positive hybridoma cells: The RSV F protein was diluted in coating buffer (0.05 mol/L, pH 9.6, PBS) to a final concentration of 1 μg/mL, and added to a 96-well plate at 100 μL/well, followed by coating at 4° C. overnight. After the coating solution was discarded, the plate was washed three times in phosphate buffered saline (PBST) and patted dry. The plate was blocked with PBST containing 2% BSA (150 μL/well), incubated at 37° C. for 2 h, washed three times in PBST, and patted dry. The fused cell supernatant, positive serum from the immunized mice (1:1000 diluted, as a positive control) and negative serum from unimmunized mice (1:1000 diluted, as a negative control) were added to corresponding wells at 100 μL/well, incubated at 37° C. for 1 h, washed three times in PBST, and patted dry. Horseradish peroxidase (HRP)-labeled goat anti-mouse IgG (1:4000 diluted, purchased from Sigma) was added to the plate at 100 μL/well, incubated at 37° C. for 1 h, washed three times in PBST, and patted dry. Tetramethylbenzidine (3,3′,5,5′-Tetramethylbenzidine, TMB) substrate was added at 100 μL/well, and allowed to develop at room temperature in the dark for 10 min. 50 μL of 2 mol/L sulfuric acid was added to each well to terminate the reaction.

The OD₄₅₀ₙₘ values of all the wells were measured on a microplate reader at a wavelength of 450 nm. A test well is defined as positive if the OD₄₅₀ₙₘ absorbance value of the test well (RSV F protein) is at least 2.1 times of the OD₄₅₀ₙₘ value of the negative control well, provided the OD₄₅₀ₙₘ of the negative serum is 0.1. Positive hybridoma cells were screened out for subsequent cloning.

Cloning of positive cell strains: Positive antibody-secreting cell wells were sampled, counted, and then diluted to 100 cells/10 mL culture medium. The diluted cell suspension was plated into a 96-well plate at 100 μL/well, and cultured in an incubator at 37° C. with 5% CO₂. After 6-7 days, clone cell formation was observed under a microscope. Wells containing single clones were marked, and the cell supernatants were collected for ELISA detection (performed as described in the fusion cell screening) to select positive monoclonal cells. The cells from positive wells were subjected to limiting dilution, and the ELISA values were measured 5-6 days after each limiting dilution. The monoclonal wells with higher OD₄₅₀ₙₘ positive values measured by ELISA were selected for further limiting dilution until all the wells of the entire 96-well plate were tested positive by ELISA. The monoclonal stable strains with high positive values were selected. Finally, three cell strains that stably secrete anti-RSV antibodies were obtained and named hybridoma cell strains F2H11, B8H12 and C5B7 respectively.

The hybridoma cell strains F2H11, B8H12, and C5B7 were deposited under accession numbers CGMCC No. 45238, CGMCC No. 45239, and CGMCC No. 45240, respectively, at the China General Microbiological Culture Collection Center (CGMCC), Institute of Microbiology, Chinese Academy of Sciences, located at Room 3, NO. 1 West Beichen Road, Chaoyang District, Beijing, China, on Jul. 14, 2022.

Preparation and purification of monoclonal antibodies from cell supernatant: The three cell strains were cultured in RPMI-1640 medium containing 15% serum in 10 cm culture dishes. When the cells were expanded to about 4×10⁷/dish, the cells were centrifuged at 800 rpm for 5 min to discard the supernatant, and transferred to a 2 L spinner flask. Serum-free culture medium was added to adjust the cell density to about 3×10⁵/mL for spinner flask culture. After 1-2 weeks of further culture, when the cell death rate reached 80%-90% (at this time, the cell density was about 1×10⁶−2×10⁶ cells/mL), the cell suspension was collected and centrifuged at 6000 rpm for 20 min to harvest the supernatant. The supernatant was purified by Protein A immunochromatographic assay. The monoclonal antibodies produced by F2H11, B8H12 and C5B7 hybridoma cells were designated as RSV101, RSV200 and RSV300, respectively.

The concentrations of the monoclonal antibodies were measured by a micro spectrophotometer, and the results showed that the concentrations of RSV101, RSV200 and RSV300 monoclonal antibodies were about 2-4 mg/mL. The monoclonal antibodies were aliquoted (100 μL/tube, at a concentration of 1 mg/mL) and stored at 4° C.-8° C.

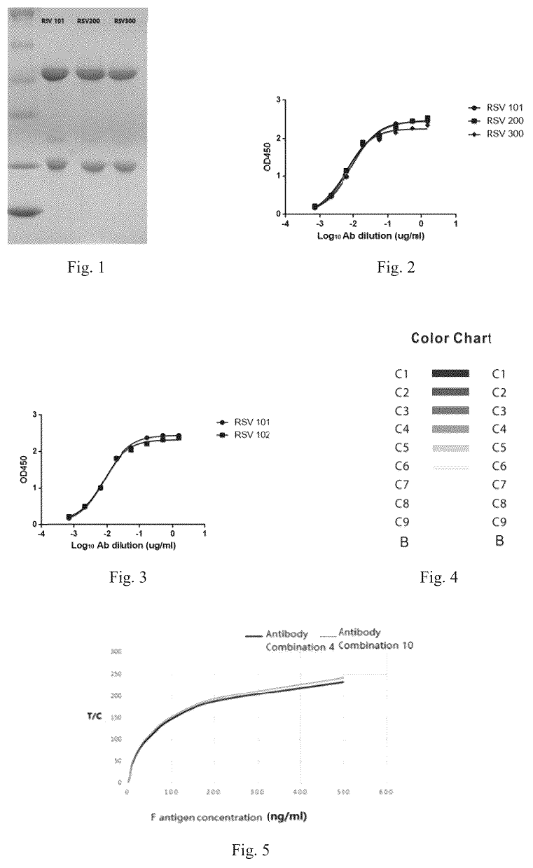

The three antibodies were identified by SDS-PAGE electrophoresis (5 μg loaded per lane), and the results are shown in FIG. 1. As can be seen from FIG. 1, the three antibodies each showed an antibody heavy chain band of about 51 KD and an antibody light chain band of about 26 KD.

Purity Analysis: The three monoclonal antibodies mentioned above were analyzed by size exclusion chromatography (SEC-HPLC). Under the conditions that ensure complete elution of all components in the tested samples, the purity percentage of the main peak was calculated by peak area normalization method. All antibodies demonstrated a purity greater than 98%.

### Example 2: Binding Ability of Antibodies to RSV F Protein

RSV F protein was diluted in 0.05 mol/L carbonate buffer (pH 9.6) to a final concentration of 1 μg/mL, and added to a 96-well microplate at 100 μL/well, and then the microplate was coated at 4° C. overnight, washed 3 times in PBST on an automatic plate washer, and patted dry. The wells were blocked with PBST containing 2% BSA at 150 μL/well, incubated at 37° C. for 2 h, washed 3 times in PBST, and patted dry. The anti-RSV F protein monoclonal antibodies RSV101, RSV200 and RSV300 were subjected to threefold serial dilutions in 0.02 M PBS buffer (pH7.4), starting from an initial concentration of 1.5 μg/mL, to generate a series of monoclonal antibody samples with different concentrations. The diluted monoclonal antibody samples were added to the above microplate at 100 μL/well, incubated at 37° C. for 1 hour, washed 3 times, and patted dry. HRP-conjugated goat anti-mouse IgG (purchased from Sigma-Aldrich) (1:4000 diluted) was added at 100 μL/well, incubated at 37° C. for 1 hour, washed 3 times in PBST, and patted dry. TMB substrate was added at 100 μL/well and allowed to develop at room temperature in the dark for 10 min. 2 mol/L sulfuric acid was added at 50 μL/well to terminate the reaction. The OD₄₅₀ₙₘ values were measured on a microplate reader, the above ELISA results were used to calculate via software analysis the EC₅₀ values of RSV101 antibody, RSV200 antibody, and RSV300 antibody, and the results are shown in FIG. 2.

It can be seen from FIG. 2 that the EC₅₀ values of RSV101, RSV200, and RSV300 binding to RSV-F (strain A) were 8.514 ng/mL, 6.459 ng/mL, and 6.035 ng/mL, respectively. The affinities between the antibodies and RSV-F were all at the nanomolar (nM) level, with RSV300 antibody exhibiting the highest affinity for RSV-F (strain A).

### Example 3: Cloning and Sequencing of Anti-RSV Antibody Variable Region Sequences

Total RNA was extracted from the above three hybridoma cell strains, and used to prepare cDNA by reverse transcription, to clone immunoglobulin sequences from the hybridoma cell strains, and determine the sequences of the variable regions of the antibodies produced by the above hybridoma cell strains.
a. RNA extraction: According to the instructions of the M5 Total RNA Extraction Kit (purchased from Beijing Juhemei Biotechnology Co., Ltd.), total RNA was extracted from the above hybridoma cell strains, and was subjected to reverse transcription immediately in the next step.
b. Reverse transcription of RNA into cDNA: The total RNA extracted in the previous step was subjected to reverse transcription according to the M5 First Strand cDNA Synthesis Kit (purchased from Beijing Juhemei Biotechnology Co., Ltd.) to obtain cDNA, which was frozen at −20° C. for future use.
c. PCR amplification and recovery of variable region sequences: The immunoglobulin heavy chain (IgH) cDNA was amplified by PCR using cDNA obtained in the previous step as a template and universal heavy chain primers Mu Ig V_{H} 5′-A and Mu IgG V_{H} 3′-2. Similarly, the immunoglobulin light chain (IgK) cDNA was amplified by PCR using light chain primers Mu IgK V_{L} 5′-A and Mu IgK V_{L} 3′-1. The PCR products were then recovered. The thermostable PfuDNA polymerase was used throughout the PCR reaction.
d. Cloning and sequencing of variable region sequences: According to the instructions of the cloning vector pTOPO-Blunt Cloning kit (purchased from Beijing Juhemei Biotechnology Co., Ltd.), the heavy-chain and light-chain variable region genes were ligated into the pTOPO vectors respectively, which were then transformed into Escherichia coli DH5α, and the positive clones were picked and submitted to Beijing Ruiboxingke Biotechnology Co., Ltd. for sequencing.

The antibody heavy-chain and light-chain variable region gene sequences from the hybridoma cell strains were analyzed, and the complementarity-determining region sequences of the heavy and light chains are shown in Table 1 below.

TABLE 1
Complementarity-determining region sequences of antibody heavy and
light chains (based on the Chothia numbering system)

| Antibody | | | |
|---|---|---|---|
| designation | CDR | Sequence | SEQ ID NO. |
| RSV101 | V_{H}CDR 1 | GYSITSDY | SEQ ID NO: 1 |
| | V_{H}CDR 2 | SYIGS | SEQ ID NO: 2 |
| | V_{H}CDR 3 | TIRNYFDY | SEQ ID NO: 3 |
| | V_{L}CDR 1 | SASQGISNYLN | SEQ ID NO: 4 |
| | V_{L}CDR 2 | DTSSLHS | SEQ ID NO: 5 |
| | V_{L}CDR 3 | QQYSNFPXT (wherein, X is C) | SEQ ID NO: 6 |
| RSV200 | V_{H}CDR 1 | GFNIKDY | SEQ ID NO: 7 |
| | V_{H}CDR 2 | DPENGN | SEQ ID NO: 8 |
| | V_{H}CDR 3 | YGTSYWFPY | SEQ ID NO: 9 |
| | V_{L}CDR 1 | KASQDINSYLS | SEQ ID NO: 10 |
| | V_{L}CDR 2 | RANRLVD | SEQ ID NO: 11 |
| | V_{L}CDR 3 | LQFDEFPYT | SEQ ID NO: 12 |
| RSV300 | V_{H}CDR 1 | GYTFSSN | SEQ ID NO: 13 |
| | V_{H}CDR 2 | LPGSGS | SEQ ID NO: 14 |
| | V_{H}CDR 3 | EELGYFDY | SEQ ID NO: 15 |
| | V_{L}CDR 1 | RASQDINNYLN | SEQ ID NO: 16 |
| | V_{L}CDR 2 | YTSRLHS | SEQ ID NO: 17 |
| | V_{L}CDR 3 | QQGSTLPPT | SEQ ID NO: 18 |

### Example 4: Preparation and Purification of Recombinant Antibodies

Recombinant antibodies were constructed, and cell strains that stably express anti-RSV F antibodies were prepared through eukaryotic expression, followed by large-scale culture and purification.

Specifically, for the V_{L} and V_{H} genes of the antibody, the recombinant antibody eukaryotic expression plasmid was constructed using the molecular cloning method and the plasmid pCDNA3.1. The eukaryotic expression plasmid was electroporated into CHO host cells, and the electroporated CHO host cells were cultured in selective medium (50 μM MSX) for 20 days. Then, the supernatant was taken for ELISA assay (using HRP-conjugated goat anti-mouse IgG as the secondary antibody, performed as described above), to screen out the cell strains that stably express the recombinant antibodies. The particular sequences of the heavy-chain and light-chain variable regions of the recombinant RSV antibodies are shown in Table 2 below.

In addition, the V_{H} CDR3 of the RSV101 antibody was mutated from QQYSNFPCT to QQYSNFPFT (QQYSNFPXT (SEQ ID NO: 6), where X is F), with the remaining sequence unchanged. The recombinant antibody eukaryotic expression vector was constructed in the same manner (also using plasmid pCDNA3.1). The expression vector was electroporated into CHO host cells, and the cell strains stably expressing the recombinant antibodies were screened out. The obtained recombinant antibody was designated as RSV 102. The particular sequences of the heavy-chain and light-chain variable regions of the recombinant RSV antibody 102 are shown in Table 2 below.

TABLE 2
Sequences of the heavy-chain and light-chain variable regions of recombinant RSV
antibodies (according to the Chothia numbering system)

| Re- | | |
|---|---|---|
| combinant | | |
| Antibody | Heavy-chain variable region | Light-chain variable region |
| RSV101′ | EVQLQQPGAELVKPGGSVKLSCKASGYSITS | DIVMTQTHKFMSTSVGDRVSLTCSASQGIS |
| | DYWVKORPGRQLEWIGSYIGSKISLTVDKP | NYLNWYQEKPGQSPKLLIYSDTSSLHSGVP |
| | SSTAYMQLSSLTSEDSAVYYCARTIRNYFDY | ARFTGTQSGTDFTFTISSVQAEDEALYFCQQ |
| | WGQGTTVTVSS | YSNFPCTFGAGTKLELK |
| | (SEQ ID NO: 19) | (SEQ ID NO: 20) |
| RSV102 | EVQLQQPGAELVKPGGSVKLSCKASGYSITS | DIVMTQTHKFMSTSVGDRVSLTCSASQGIS |
| | DYWVKQRPGRQLEWIGSYIGSKISLTVDKP | NYLNWYQEKPGQSPKLLIYSDTSSLHSGVP |
| | SSTAYMQLSSLTSEDSAVYYCARTIRNYFDY | ARFTGTQSGTDFTFTISSVQAEDEALYFCQQ |
| | WGQGTTVTVSS | YSNFPFTFGAGTKLELK |
| | (SEQ ID NO: 19) | (SEQ ID NO: 28) |
| RSV200′ | EVQLQQPGAELVKPGGSVKLSCKASGFNIK | DIVMTQTHKFMSTSVGDRVSLTCKASQDIN |
| | DYWVKQRPGRQLEWIGDPENGNKISLTVD | SYLSWYQEKPGQSPKLLIYSRANRLVDGVP |
| | KPSSTAYMQLSSLTSEDSAVYYCARYGTSYW | ARFTGTQSGTDFTFTISSVQAEDEALYFCLQ |
| | FPYWGQGTTVTVSS | FDEFPYTFGAGTKLELK |
| | (SEQ ID NO: 21) | (SEQ ID NO: 22) |
| RSV300′ | EVQLQQPGAELVKPGGSVKLSCKASGYTFS | DIVMTQTHKFMSTSVGDRVSLTCRASQDI |
| | SNWVKQRPGRQLEWIGLPGSGSKISLTVDK | NNYLNWYQEKPGQSPKLLIYSYTSRLHSGV |
| | PSSTAYMQLSSLTSEDSAVYYCAREELGYFD | PARFTGTQSGTDFTFTISSVQAEDEALYFCQ |
| | YWGQGTTVTVSS | QGSTLPPTFGAGTKLELK |
| | (SEQ ID NO: 23) | (SEQ ID NO: 24) |

The screened stably transformed cell strains were subjected to large scale culture using the roller bottle technique for recombinant antibody production. The cells were inoculated in roller bottles at (0.2−0.3)×10⁶ cells/mL using culture medium (Vega CHO), with 300 mL culture medium (Vega CHO) per 1 L roller bottle. The number of inoculated roller bottles was determined according to production needs. The roller bottles inoculated with cells were placed in a cell roller bottle machine and cultured in an incubator at 900 rpm and 37° C. with 5% CO₂. After 7-9 days of culture, samples were taken for observation under a microscope. When the cell viability dropped to less than 50%, the supernatant was harvested via centrifugation, and subjected to affinity purification via Protein A affinity chromatography to obtain recombinant monoclonal antibodies.

In addition, according to the method described in Example 2, the binding ability of recombinant antibodies RSV101′, RSV102, RSV200′ and RSV300′ to RSV F (strain A) protein was detected. The results showed that the EC₅₀ values of these antibodies binding to RSV F protein were 8.4 ng/mL, 7.9 ng/mL, 6.4 ng/mL and 6.0 ng/mL, respectively. The affinity between the antibodies and RSV F protein was at the nM level. The results of RSV101′ and RSV102 are shown in FIG. 3.

### Example 5: Colored Latex Microsphere-Based Immunochromatographic Assay

The anti-RSV monoclonal antibodies of the present disclosure were evaluated by colored latex microsphere-based immunochromatographic assay.

Labeling of Latex Microspheres with Recombinant Antibody:
1. Microsphere washing: 1 mg of microspheres was added to an amount of 0.1 M 4-morpholineethanesulfonic acid (MES) buffer (pH 6.0), mixed well, and centrifuged at 20,000 rpm for 20 min, and the supernatant was removed.
2. Activation: The microspheres were suspended in a volume of MES buffer (pH 6.0), mixed by ultrasonication, added with 12 μg 1-ethyl-(3-dimethylaminopropyl) carbodiimide (EDC) and 12 μg N-hydroxysuccinimide (NHS), allowed to react at 37° C. for 15 min, with mixing every 5 min, and then centrifuged at 20,000 rpm for 20 min, and the supernatant was removed.
3. Conjugation: The microspheres were suspended in a volume of 50 mM boric acid buffer (pH=8.0), mixed by ultrasonication, added with 0.2 mg of anti-RSV F monoclonal antibody a listed in Table 3 below, mixed well, and allowed to react at 37° C. overnight.
4. Blocking: The microspheres were blocked with 1 mL BSA (5%) for 1 h, and then centrifuged at 20,000 rpm for 20 min, and the supernatant was removed.
5. Washing: The microspheres were washed twice in 0.1 M Tris-HCl (pH8.5), and then centrifuged at 20000 rpm for 20 min, and the supernatant was removed.
6. Storage: The microspheres were resuspended in 0.5 mL of 25 mM MES (pH=7.4), diluted 50-fold in 25 mM MES (pH=7.4, containing 1% BSA and 0.1% Tween 20), freeze-dried and sealed for later use.

Coating of nitrocellulose membrane (NC membrane) with recombinant antibody: The anti-RSV monoclonal antibody b (listed in Table 3 below) as the coating antibody was diluted in 10 mM PBS (pH 7.4, containing 2% sucrose) to a final concentration of 1.0 mg/mL, and coated onto the Sartorius 140 NC membrane as the test line (T line). After incubation at 37° C. overnight, the membrane was then sealed for future use.

Preparation of sample pad: The sample pad was treated with 10 mM PBS (pH 7.4), lyophilized, and stored for later use.

Assembly of immunochromatographic rapid test strip: The above-mentioned antibody-labeled red latex microspheres, the antibody-coated NC membrane, the sample pad, the absorbent pad, and the polyester backing plate were assembled into an immunochromatographic rapid test strip using standard procedures.

TABLE 3
Anti-RSV monoclonal antibodies a and b used in this example

| Antibody Combination | | |
|---|---|---|
| NO. | Labeling antibody a | Coating antibody b |
| 1 | RSV101′ | RSV101′ |
| 2 | RSV101′ | RSV102 |
| 3 | RSV101′ | RSV200′ |
| 4 | RSV101′ | RSV300′ |
| 5 | RSV102 | RSV101′ |
| 6 | RSV102 | RSV102 |
| 7 | RSV102 | RSV200′ |
| 8 | RSV102 | RSV300′ |
| 9 | RSV200′ | RSV101′ |
| 10 | RSV200′ | RSV102 |
| 11 | RSV200′ | RSV200′ |
| 12 | RSV200′ | RSV300′ |
| 13 | RSV300′ | RSV101′ |
| 14 | RSV300′ | RSV102′ |
| 15 | RSV300′ | RSV200′ |
| 16 | RSV300′ | RSV300′ |

Immunochromatographic assay: Samples of virus culture at different dilutions (1:100, 1:1000, and 1:10000) and samples containing the recombinant antigen at different concentrations (1 ng/mL, 10 ng/mL, and 100 ng/mL) were tested. Specifically, 80 μL of the sample to be tested was applied to the rapid test strip. After incubation at room temperature for 15 minutes, the results were observed and interpreted visually. The color intensity of the test line on the strip indicates the activity of the antigen in the sample binding to the antibody. The results are shown in Table 4, with the RSV latex chromatographic test strip from manufacturer A as a control. The colored T-line band was compared with the reference color chart to match the closest color, and the color number corresponding to the closest color was used to indicate the activity of the product. As shown in FIG. 4, the color chart is divided into nine levels from C2 to C9, among which C represents the strongest positive signal, indicating the highest unit activity, the middle levels decrease gradientally, and C9 represents the weakest positive signal, indicating the lowest unit activity, and B in the figure represents blank.

TABLE 4
Results of immunochromatographic assays based on colored latex microspheres

| | Virus Culture | | | Recombinant Antigen | | |
|---|---|---|---|---|---|---|
| Test Results | 1:100 | 1:1000 | 1:10000 | 100 | 10 | 1 |
| Antibody Combination 1 | C6 | C9 | B | C5 | C8 | B |
| Antibody Combination 2 | C2 | C5 | C8 | C2 | C4 | C7 |
| Antibody Combination 3 | C2 | C4 | C8 | C2 | C4 | C7 |
| Antibody Combination 4 | C2 | C5 | C7 | C2 | C4 | C6 |
| Antibody Combination 5 | C2 | C4 | C8 | C2 | C4 | C7 |
| Antibody Combination 6 | C7 | C9 | B | C6 | C9 | B |
| Antibody Combination 7 | C2 | C4 | C8 | C2 | C4 | C7 |
| Antibody Combination 8 | C2 | C5 | C7 | C2 | C4 | C6 |
| Antibody Combination 9 | C2 | C5 | C8 | C2 | C4 | C7 |
| Antibody Combination 10 | C2 | C4 | C7 | C2 | C4 | C6 |
| Antibody Combination 11 | C6 | C9 | B | C5 | C9 | B |
| Antibody Combination 12 | C2 | C4 | C8 | C2 | C4 | C7 |
| Antibody Combination 13 | C2 | C5 | C7 | C2 | C4 | C6 |
| Antibody Combination 14 | C2 | C5 | C7 | C2 | C4 | C6 |
| Antibody Combination 15 | C2 | C5 | C8 | C2 | C4 | C7 |
| Antibody Combination 16 | C7 | C9 | B | C6 | C9 | B |
| RSV latex chromatographic | C3 | C6 | C9 | C3 | C5 | C8 |
| test strip from manufacturer A | | | | | | |
| (Control) | | | | | | |

The results showed that, in the colored latex microsphere-based immunochromatographic assays, when different labeling antibodies and coating antibodies were used, whether for the virus culture or the recombinant antigen, the sensitivity was one to two levels higher than that of the similar product from manufacturer A, and reached the nanoscale level, among which Antibody Combinations 4, 10, 13 and 14 showed the highest sensitivity.

### Example 6: Colloidal Gold Immunochromatographic Assay

Preparation of colloidal gold: 200 mL of ultrapure water was added to a conical flask, heated to boiling, added with 1 mL of 2% chloroauric acid (Sigma-Aldrich, Catalog No.: 16961-25-4) solution, and added with 1 mL of 2% trisodium citrate (Sigma-Aldrich, Catalog No.: 6132-04-3) aqueous solution immediately after boiling. The mixture was allowed to boil with stirring for 10 minutes, and then to cool naturally for later use.

Labeling of colloidal gold: 10 mL of the above colloidal gold was put into a beaker, 120 μL of 0.2 M K₂CO₃ was added to adjust the pH to 7.0 while stirring, and stirring continued for 10 seconds; 100 g of anti-RSV monoclonal antibody a (the labeling antibody, as listed in Table 3 above) was added and stirred for 5 minutes; 0.1 mL of 10% BSA was added and stirred for 5 minutes; 0.1 mL 10% BSA was added and stirred for 5 minutes; the mixture was centrifuged at 12000 g for 10 minutes, the supernatant was discarded, and the pellet was diluted in colloidal gold dilution buffer (10 mM PB, 150 mM NaCl, 0.2% BSA, 0.1% TritonX-100, 3% Sucrose, and 0.01% Proclin 300) to a final volume of 1 mL, as the anti-RSV monoclonal antibody-colloidal gold complex.

Preparation of colloidal gold conjugate pad: The colloidal gold complex was diluted 10-fold in colloidal gold dilution buffer, and used to soak the glass fiber pad (Shanghai Jinbiao Company), and the soaked pad was freeze-dried as the colloidal gold conjugate pad.

Coating of nitrocellulose membrane (NC membrane): The anti-RSV monoclonal antibody b (the coating antibody, listed in Table 3 above) was diluted to 1.5 mg/mL to prepare the working solution for test line. The working solution was dispensed onto corresponding positions of the nitrocellulose membrane (Millipore, catalog number: HF135002) by using a membrane dispenser, and the membrane was dried at 50° C. for 1 hour and stored for later use.

Assembly of colloidal gold immunochromatographic test strip: The above-mentioned colloidal gold conjugate pad, the antibody-coated nitrocellulose membrane, the absorbent pad, the polyester backing plate, and the sample pad were assembled into a colloidal gold immunochromatographic test strip.

Sensitivity Determination: The samples of virus culture at different dilutions and the recombinant antigen at different concentrations were tested. Specifically, 80 μL of the sample to be tested was applied to the sample pad. After incubation at room temperature for 15-30 minutes, results were interpreted. The color intensity of the test line of the strip indicates the activity of the antigen in the sample binding to the antibody. The colored T-line band on the colloidal gold test strip was compared with the reference color chart to match the closest color, and the color number corresponding to the closest color was used to indicate the activity of the product. The results are shown in Table 5 below, with the RSV colloidal gold chromatographic test strip from manufacturer A as a control. The results showed that, the antibody combinations of the present disclosure showed sensitivity comparable to or one to two levels higher than that of the RSV colloidal gold chromatographic test strip from manufacturer A, among which antibody combinations 4, 10 and 13 showed the highest sensitivity.

TABLE 5
Results of immunochromatographic assay based on
colloidal gold (ng/ml for recombinant antigen)

| | Virus Culture | | | Recombinant Antigen | | |
|---|---|---|---|---|---|---|
| Test Results | 1:100 | 1:1000 | 1:10000 | 100 | 10 | 1 |
| Antibody Combination 1 | C7 | C9 | B | C6 | C9 | B |
| Antibody Combination 2 | C3 | C6 | C9 | C3 | C5 | C8 |
| Antibody Combination 3 | C3 | C5 | C9 | C3 | C5 | C8 |
| Antibody Combination 4 | C3 | C6 | C8 | C3 | C5 | C7 |
| Antibody Combination 5 | C4 | C6 | C9 | C3 | C6 | C9 |
| Antibody Combination 6 | C7 | C9 | B | C6 | C9 | B |
| Antibody Combination 7 | C3 | C5 | C9 | C3 | C5 | C8 |
| Antibody Combination 8 | C3 | C6 | C8 | C3 | C6 | C9 |
| Antibody Combination 9 | C3 | C6 | C9 | C3 | C6 | C8 |
| Antibody Combination 10 | C3 | C5 | C8 | C3 | C5 | C7 |
| Antibody Combination 11 | C7 | C9 | B | C6 | C9 | B |
| Antibody Combination 12 | C3 | C5 | C9 | C3 | C5 | C8 |
| Antibody Combination 13 | C3 | C5 | C8 | C3 | C5 | C7 |
| Antibody Combination 14 | C3 | C6 | C8 | C3 | C5 | C8 |
| Antibody Combination 15 | C3 | C6 | C9 | C3 | C5 | C8 |
| Antibody Combination 16 | C7 | C9 | B | C6 | C9 | B |
| RSV colloidal gold | C4 | C7 | C9 | C4 | C6 | C9 |
| chromatography reagent from | | | | | | |
| manufacturer A (Control) | | | | | | |

Specificity Determination: 30 nasopharyngeal swab samples from healthy people were tested by using the test strips prepared with four recombinant antibodies of the present disclosure, with the RSV colloidal gold immunochromatographic test strip from manufacture A as a control. The results showed that, similar to the test strip of manufacturer A, the test strips prepared with four recombinant antibodies of the present disclosure had no false positive results and achieved a specificity of 100%.

### Example 7: Fluorescent Microsphere Immunochromatographic Assay

The recombinant antibodies of the present disclosure were used to coat NC membrane (as the coating antibody) and time-resolved fluorescent microspheres (as the labeling antibody) to prepare the chromatographic product. The procedures were as follows:
1. Labeling of time-resolved fluorescent microspheres: 1 mg of microspheres were added to an amount of 0.1 M MES buffer solution (pH 6.5), mixed well and centrifuged at 20,000 rpm for 20 min, and the supernatant was removed; the pellet was suspended in a volume of MES buffer solution (pH 6.5), mixed by ultrasonication, and added with 20 μg EDC and 40 μg NHS, and the mixture was allowed to react at room temperature for 30 min, with mixing every 5 min, and then centrifuged at 20,000 rpm for 20 min, and the supernatant was discarded; the pellet was suspended in a volume of 50 mM boric acid buffer (pH=8.0), mixed by ultrasonication, and added with 0.05-0.1 mg of anti-RSV monoclonal antibody a listed in Table 3 above, mixed well and allowed to react at room temperature for 2 hours; the microspheres were blocked with 1 mL 100 mM Tris-HCl (pH8.5, containing 1% BSA) for 2 hours, and centrifuged at 20000 rpm for 20 min, and the supernatant was discarded; the pellet was washed twice in 0.1M Tris-HCl (pH8.5), and centrifuged at 20,000 rpm for 20 min, and the supernatant was discarded; the pellet was resuspended in 0.5 mL of 25 mM MES (pH=7.4), diluted 50-fold in 25 mM MES (pH=7.4, containing 1% BSA and 0.1% Tween 20), freeze-dried, sealed and stored for later use.
2. Coating of NC membrane: Anti-RSV monoclonal antibody b listed in Table 3 above was diluted in 10 mM PBS (pH 7.4, containing 2% sucrose) to a final concentration of 1.0 mg/mL, and coated onto Sartorius 140 NC membrane as the test line (T line). After incubation at 37° C. overnight, the membranes were sealed and stored for later use.
3. Preparation of sample pad: The sample pad was treated with 10 mM PBS (pH 7.4), freeze-dried and stored for later use.
4. Assembly and Testing: the above-mentioned antibody-labeled time-resolved fluorescent microspheres, the antibody-coated NC membrane, and the sample pad were assembled into an immunochromatographic rapid test strip. The strip was applied with 80 IL of a clinical sample, placed at room temperature for 15 minutes, and then detected by an immunofluorescence analyzer.

### Antibody Sensitivity to F Protein Recombinant Antigen:

First, Antibody Combinations 4 and 10 were tested for sensitivity to F protein recombinant antigen at different concentrations (0.1-500 ng/mL), and the results are shown in Table 6 and FIG. 5 below. As shown in Table 6 below, the test results of Antibody Combination 4 for F protein recombinant antigen showed that, when the antigen concentration was 0.1 ng/ml, the T/C value was more than twice the T/C value (0.52) of the test groups without F protein, which confirmed that, in the fluorescent microsphere immunochromatographic system, the sensitivity of the recombinant antibody to F protein recombinant antigen reached 0.1 ng/mL. The same test was performed using RSV N protein recombinant antigen as a negative control, and it was found that the T/C value did not change significantly with the increase in N protein concentration.

TABLE 6
Sensitivity test results of Antibody Combination
4 to F protein recombinant antigen

| Recombinant | | | | |
|---|---|---|---|---|
| antigen | | | | |
| concentration | | | | |
| (ng/mL) | T | C | T/C | Mean |
| 0 | 5421 | 10350 | 0.52 | 0.52 |
| | 6726 | 12920 | 0.52 | |
| 0.1 | 12370 | 11561 | 1.07 | 1.05 |
| | 11687 | 11347 | 1.03 | |
| 0.25 | 14372 | 12180 | 1.18 | 1.20 |
| | 13627 | 11170 | 1.22 | |
| 0.5 | 16005 | 11005 | 1.45 | 1.44 |
| | 16972 | 11930 | 1.42 | |
| 1.0 | 19417 | 10040 | 1.93 | 1.96 |
| | 20931 | 10500 | 1.99 | |
| 2.5 | 59661 | 12135 | 4.92 | 4.94 |
| | 62349 | 12580 | 4.96 | |
| 5.0 | 137009 | 11595 | 11.82 | 11.56 |
| | 137499 | 12160 | 11.31 | |
| 10.0 | 506089 | 11855 | 42.69 | 41.34 |
| | 480202 | 12010 | 39.98 | |
| 25.0 | 922015 | 12275 | 75.11 | 75.54 |
| | 884746 | 11645 | 75.98 | |
| 50 | 1279119 | 12050 | 106.15 | 106.03 |
| | 1340169 | 12655 | 105.90 | |
| 100 | 1986342 | 13680 | 145.20 | 146.93 |
| | 1989011 | 13380 | 148.66 | |
| 200 | 2386790 | 12940 | 184.45 | 187.42 |
| | 2400923 | 12610 | 190.40 | |
| 500 | 2986790 | 12940 | 230.82 | 233.11 |
| | 3031952 | 12880 | 235.4 | |

Next, the test results of Antibody Combinations 2, 4, 8, 10, and 15 were compared with those of the RSV latex chromatographic test strip from manufacturer A for F protein recombinant antigen at different concentrations, as shown in Table 7. As compared with the RSV latex chromatographic test strip from manufacturer A, the antibody combinations of the present disclosure could detect F protein recombinant antigen at a concentration of 0.1 ng/mL in the fluorescent microsphere immunochromatographic assay, while the result of the latex test strip of manufacturer A was negative at this concentration, which demonstrates superior sensitivity of the monoclonal antibody of the present disclosure.

TABLE 7
Antibody sensitivity test results to virus
culture and F protein recombinant antigen

| | | F protein recombinant antigen (ng/mL) | | | |
|---|---|---|---|---|---|
| Test Results | Dilution | 100 | 10 | 1 | 0.1 |
| Antibody Combination | 0.51 | 141.83 | 38.51 | 1.81 | 1.01 |
| (T/C) 2 | | | | | |
| Antibody Combination | 0.52 | 146.93 | 41.34 | 1.96 | 1.05 |
| (T/C) 4 | | | | | |
| Antibody Combination | 0.51 | 147.15 | 40.51 | 2.07 | 1.06 |
| (T/C) 8 | | | | | |
| Antibody Combination | 0.51 | 151.23 | 45.23 | 2.21 | 1.13 |
| (T/C) 10 | | | | | |
| Antibody Combination | 0.51 | 139.77 | 39.23 | 2.07 | 1.07 |
| (T/C) 15 | | | | | |
| RSV latex | B | C3 | C5 | C8 | B |
| chromatography | | | | | |
| reagent from | | | | | |
| manufacturer A | | | | | |

Sensitivity of antibodies to virus culture: The sensitivity of the antibodies to virus culture in a dilution range of 1:100 to 1:20000 was tested, and the results are shown in Table 8. As compared with the RSV latex chromatographic test strip of manufacturer A, the antibody combinations of the present disclosure could detect virus culture at a dilution of 1:20000 in the fluorescent chromatographic assay, while the result of the latex test strip of manufacturer A was negative at a dilution of 1:20000, which demonstrates superior sensitivity of the monoclonal antibody of the present disclosure.

TABLE 8
Sensitivity test results of the antibodies
of the present disclosure to virus culture

| | | Virus culture | | | |
|---|---|---|---|---|---|
| Test Result | Dilution | 1:100 | 1:1000 | 1:10000 | 1:20000 |
| Antibody Combination | 0.51 | 131.16 | 36.56 | 1.79 | 1.03 |
| (T/C) 2 | | | | | |
| Antibody Combination | 0.51 | 133.77 | 37.18 | 1.87 | 1.08 |
| (T/C) 4 | | | | | |
| Antibody Combination | 0.51 | 141.06 | 36.55 | 1.96 | 1.05 |
| (T/C) 8 | | | | | |
| Antibody Combination | 0.51 | 137.46 | 40.56 | 1.89 | 1.06 |
| (T/C) 10 | | | | | |
| Antibody Combination | 0.51 | 130.51 | 35.97 | 1.76 | 1.04 |
| (T/C) 15 | | | | | |
| RSV latex | B | C3 | C6 | C9 | B |
| chromatography | | | | | |
| reagent from | | | | | |
| manufacturer A | | | | | |

1.-15. (canceled)

16. An anti-respiratory syncytial virus (RSV) monoclonal antibody or an antigen-binding fragment thereof, comprising a heavy-chain variable region and a light-chain variable region, wherein:
1) the heavy-chain variable region comprises heavy-chain complementarity-determining regions V_{H} CDR1, V_{H} CDR2 and V_{H} CDR3 with amino acid sequences shown in SEQ ID NO: 1-3, respectively, and the light-chain variable region comprises light-chain complementarity-determining regions V_{L} CDR1, V_{L} CDR2 and V_{L} CDR3 with amino acid sequences shown in SEQ ID NO: 4-6, respectively, wherein the sequence shown in SEQ ID NO: 6 is QQYSNFPXT, wherein X is C or F;
2) the heavy-chain variable region comprises heavy-chain complementarity-determining regions V_{H} CDR1, V_{H} CDR2 and V_{H} CDR3 with amino acid sequences shown in SEQ ID NO: 7-9, respectively, and the light-chain variable region comprises light-chain complementarity-determining regions V_{L} CDR1, V_{L} CDR2 and V_{L} CDR3 with amino acid sequences shown in SEQ ID NO: 10-12, respectively; or
3) the heavy-chain variable region comprises heavy-chain complementarity-determining regions V_{H} CDR1, V_{H} CDR2 and V_{H} CDR3 with amino acid sequences shown in SEQ ID NO: 13-15, respectively, and the light-chain variable region comprises light-chain complementarity-determining regions V_{L} CDR1, V_{L} CDR2 and V_{L} CDR3 with amino acid sequences shown in SEQ ID NO: 16-18, respectively.

17. The anti-respiratory syncytial virus (RSV) monoclonal antibody or the antigen-binding fragment thereof according to claim 16, wherein the heavy-chain variable region comprises heavy-chain complementarity-determining regions V_{H} CDR1, V_{H} CDR2 and V_{H} CDR3 with amino acid sequences shown in SEQ ID NO: 1-3, respectively, and the light-chain variable region comprises light-chain complementarity-determining regions V_{L} CDR1, V_{L} CDR2 and V_{L} CDR3 with amino acid sequences shown in SEQ ID NO: 4-6, respectively, wherein the sequence shown in SEQ ID NO: 6 is QQYSNFPXT, wherein X is F.

18. The anti-respiratory syncytial virus (RSV) monoclonal antibody or the antigen-binding fragment thereof according to claim 16, wherein:
the heavy-chain variable region comprises an amino acid sequence shown in SEQ ID NO: 19, or a sequence having more than 80% identity with the amino acid sequence shown in SEQ ID NO: 19, and the light-chain variable region comprises an amino acid sequence shown in SEQ ID NO: 20, or a sequence having more than 80%, identity with the amino acid sequence shown in SEQ ID NO: 20;
the heavy-chain variable region comprises an amino acid sequence shown in SEQ ID NO: 19, or a sequence having more than 80% identity with the amino acid sequence shown in SEQ ID NO: 19, and the light-chain variable region comprises an amino acid sequence shown in SEQ ID NO: 28, or a sequence having more than 80% identity with the amino acid sequence shown in SEQ ID NO: 28;
the heavy-chain variable region comprises an amino acid sequence shown in SEQ ID NO: 21, or a sequence having more than 80% identity with an amino acid sequence shown in SEQ ID NO: 21, and the light-chain variable region comprises the amino acid sequence shown in SEQ ID NO: 22, or a sequence having more than 80% identity with the amino acid sequence shown in SEQ ID NO: 22; or
the heavy-chain variable region comprises an amino acid sequence shown in SEQ ID NO: 23, or a sequence having more than 80% identity with the amino acid sequence shown in SEQ ID NO: 23, and the light-chain variable region comprises an amino acid sequence shown in SEQ ID NO: 24, or a sequence having more than 80% identity with the amino acid sequence shown in SEQ ID NO: 24.

19. A hybridoma cell, which is deposited under accession number CGMCC No. 45238, CGMCC No. 45239 or CGMCC No. 45240 in the China General Microbiological Culture Collection Center (CGMCC), Institute of Microbiology, Chinese Academy of Sciences, located at Room 3, NO. 1 West Beichen Road, Chaoyang District, Beijing, China, on Jul. 14, 2022.

20. A nucleic acid molecule encoding the anti-RSV monoclonal antibody or the antigen-binding fragment thereof according to claim 16.

21. A vector comprising the nucleic acid molecule according to claim 20.

22. The vector according to claim 21, wherein the vector is a plasmid vector.

23. The vector according to claim 22, wherein the plasmid vector is any one of pEE12, pCAGGS, pTOPO, pcDNA such as pCDNA3.1, pTT, pTT3, pEFBOS, pBV, pJV and pBJ.

24. An expression cell, comprising the vector according to claim 21.

25. The expression cell according to claim 24, wherein the expressing cell is a mammalian cell.

26. The expression cells according to claim 25, wherein the mammalian cell is Chinese hamster ovary cells, baby hamster kidney cells, monkey kidney cells, mouse thymoma cells, or human embryonic kidney cells.

27. Use of the anti-respiratory syncytial virus (RSV) monoclonal antibody or the antigen-binding fragment thereof according to claim 16 for detecting respiratory syncytial virus (RSV).

28. The use according to claim 27, wherein the detecting respiratory syncytial virus (RSV) comprises diagnosis of respiratory syncytial virus (RSV) infection.

29. The use according to claim 27, wherein the detecting is performed by an immunochromatographic assay, an enzyme-linked antibody assay (ELISA), a chemiluminescent assay, or an electrochemiluminescent assay.

30. The use according to claim 29, wherein the immunochromatographic assay comprises fluorescent microsphere immunochromatographic assays, colloidal gold immunochromatographic assays, colored latex microsphere-based immunochromatographic assays, time-resolved fluorescent microsphere immunochromatographic assays, magnetic microsphere immunochromatographic assays or quantum dot immunochromatographic assays.

31. The use according to claim 29, wherein the ELISA is in a direct, indirect, sandwich method or competitive format.

32. A method for detecting respiratory syncytial virus (RSV), comprising a step of using the anti-respiratory syncytial virus (RSV) monoclonal antibody or the antigen-binding fragment thereof according to claim 16.

33. The method according to claim 32, the detecting respiratory syncytial virus (RSV) comprises diagnosis of respiratory syncytial virus (RSV) infection.

34. A method for diagnosing respiratory syncytial virus (RSV) infection, comprising a step of testing a sample from a subject with the anti-respiratory syncytial virus (RSV) monoclonal antibody or the antigen-binding fragment thereof according to claim 16.

35. A kit detecting respiratory syncytial virus (RSV), comprising at least one anti-RSV monoclonal antibody or the antigen-binding fragment thereof according to claim 16, and instructions for use to guide the detection of respiratory syncytial virus (RSV).
